(19) United States
(12) Patent Application Publication
(10) Pub. No.: US 2008/0051903 A1
(43) Pub. Date: Feb. 28, 2008

(54) NUCLEUS PULPOSUS IMPLANT

(76) Inventor: James DWYER, Nashanic Station, NJ (US)

Correspondence Address: KENYON & KENYON LLP, 1500 K STREET N.W., SUITE 700, WASHINGTON, DC, 20005, US

(21) Appl. No.: 11/846,124

(22) Filed: Aug. 28, 2007

Related U.S. Application Data
(60) Provisional application No. 60/840,464, filed on Aug. 28, 2006.

(51) Int. Cl. A61F 2/44

(52) U.S. Cl. 623/17.16

(57) ABSTRACT

A nucleus pulposus implant that can be converted to an interbody fusion cage is provided. Kits including the nucleus pulposus implant and other delivery components are also provided. Methods of delivering the implant into a damaged disc space are also provided.

### CROSS-REFERENCE TO RELATED APPLICATIONS

The present application claims priority to U.S. Provisional Application No. 60/840,464, filed on Aug. 28, 2006, which is incorporated by reference herein.

### TECHNICAL FIELD

The present invention relates to an implant for replacing the nucleus pulposus of an intervertebral disc.

### BACKGROUND

The intervertebral disc consists of two distinct regions: the outer series of concentric lamellae of organized collagen fibrils, known collectively as the annulus fibrosus, and the inner nucleus pulposus having a more random collagen organization and an abundance of aggregating proteoglycans. Degeneration and age-related changes in macroscopic, histologic and biochemical composition and structure of the annulus fibrosus and of the nucleus pulposus have been widely reported. In fact, degenerate disc disease account for a significant amount of disability in the United States and world population, accounting for approximately sixty billion dollars a year of cost and significant disability in people under the age of forty. In fact, degenerative disc disease accounts for eighty percent of all adults suffer from severe back pain, causing disability at one point in their life. There are numerous surgical treatment options for painful degenerative disc disease that have ranged in the past from interbody fusions and recently to total disc replacement. However, total disc replacement and lumbar fusion are two very invasive and involved, lengthy, procedures, which have significant morbidity and are also associated with a significant amount of postoperative disability. Recovery from a disc replacement can be anywhere from 12 to 50 weeks and recovery from a fusion can be anywhere from one year to two years. These approaches require both anterior and posterior approaches to the spine, often requiring significantly invasive surgery involving working around the greater vessels, including the iliac artery and veins, the vena cava and aorta.

Therefore, a need exists for a less invasive approach for treating degenerative disc disease and other pathological conditions that affect the spine.

### SUMMARY OF THE INVENTION

In an embodiment, the present invention provides a nucleus pulposus implant comprising a sterile kidney-shaped body defining at least one opening on a lateral face of the body, the opening in fluid communication with an internal cavity. The lateral face is generally parallel to the horizontal plane of the human body when the implant is in an inserted position in the intervertebral intra-nuclear space.

In another embodiment, the present invention provides a nucleus pulposus implant comprising a sterile kidney-shaped body defining at least one opening extending therethrough between opposing lateral faces of the body. The opposing lateral faces are generally parallel to the horizontal plane of the human body when the implant is in an inserted position in the intervertebral intra-nuclear space.

### BRIEF DESCRIPTION OF THE DRAWINGS

FIG. 1 is a perspective view of an implant according to an embodiment of the present invention.

FIG. 2 is a cross-sectional view of the implant of FIG. 1 according to an embodiment of the present invention.

FIG. 3 is a side view of a implant positioned on a trough according to an embodiment of the present invention.

FIG. 4 is a top view of a trough according to an embodiment of the present invention that is anchored into a disc space.

FIG. 5 is a perspective view of an intra-discal distraction device according to an embodiment of the present invention.

FIG. 6 is a view of the separate components of an intra-discal distraction device according to an embodiment of the present invention.

FIG. 7 is a schematic illustration of an intra-discal distraction device in a closed position inserted into the intervertebral space.

FIG. 8 is a schematic illustration of the intra-discal distraction device of FIG. 7 in an open position.

### DETAILED DESCRIPTION OF THE INVENTION

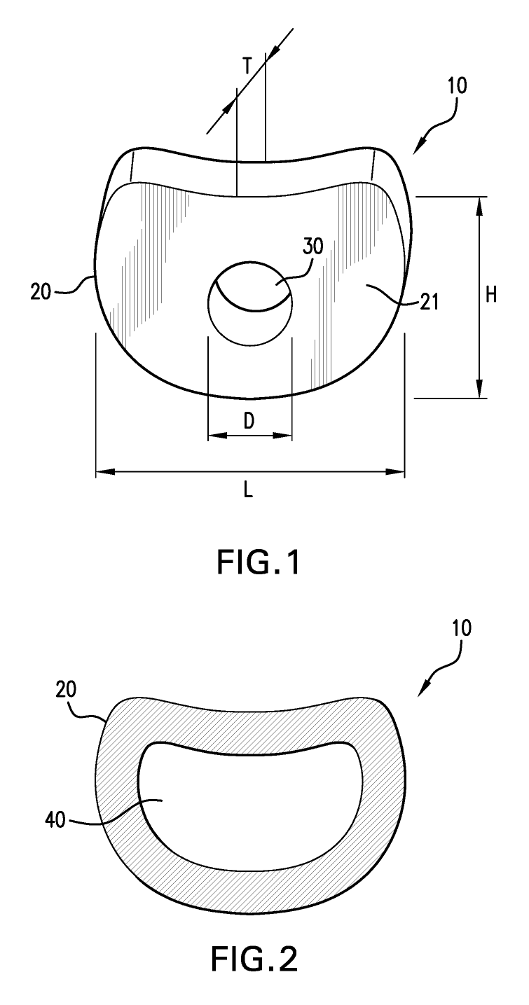

Referring to FIG. 1, in an embodiment, the present invention provides a nucleus pulposus implant 10 comprising a sterile kidney-shaped body 20 having a lateral face 21 defining at least one opening 30 in fluid communication with an internal cavity 40. When implant 10 is completely inserted into the intervertebral intra-nuclear space during the surgical procedure (i.e. in an “inserted position”), lateral face 21 is generally parallel to the horizontal plane of the human body. As is known in the art, the horizontal plane is in the reference to the human body in an upright position. Alternatively, body 20 can define an opening that extends all the way through opposing lateral faces of body 20. In certain embodiments, body 20 defines a plurality of openings 30. Internal cavity 40 is illustrated in FIG. 2, which is a cross-sectional depiction of the implant of FIG. 1. Internal cavity 40 is configured to hold bone or bone morphogenetic protein (BMP) if desired. For example, if it is desired to revise the nucleus pulposus implant into an interbody fusion cage, bone can be placed in cavity 40 to facilitate fusion between the vertebrae adjacent to the nucleus pulposus implant. Opening 30 allows bone to form around and through body 20 thereby connecting the two vertebral endplates with solid bone when the implant is implanted in the intervertebral intra-nuclear space. Specifically, opening 30 allows a bone graft to be in contact with the bony surface of adjacent vertebrae endplates. This ensures that the bone graft unites with the vertebrae, forming a solid fusion.

Regarding the dimensions of an implant of the present invention, in certain embodiments, the height H of the implant is between about 8 millimeters (mm) and 13 mm. In certain embodiments, the thickness T is between about 13 and 18 mm and preferably 15 mm. In certain embodiments, the length L is between about 22 and 27 mm and preferably 25 mm. In certain embodiments, the diameter D of opening 30 is between about 8 mm and 10 mm.

Some suitable materials for an implant according to the present invention include sterile biocompatible materials such as metallic materials and polymeric materials. Non-limiting examples of metallic materials include titanium, titanium alloys, chrome cobalt, stainless steel, or combinations thereof. Non-limiting examples of polymeric materials include high-molecular weight polyethylene, polyether ketone, polycarbonate urethane, or combinations thereof. In a preferred embodiment, the material is one that elastically deforms rather than plastically deforms. In an embodiment, the body of the implant is fabricated from a material that has elastic properties substantially equivalent to the natural elastic properties of the human body's nucleus pulposus. In certain embodiments, the implant is fabricated from bone. Preferably, the implant is fabricated from a material that has a Young's modulus from 0.5 to 100 MPa and more preferably 3 MPa. In certain embodiments, the implant is biodegradable.

Portions of the body of the implant may contain a radio-opacifying agent within their structures to facilitate viewing the implant during and/or after the implant is implanted. Non-limiting examples of radio-opacifying agents are bismuth subcarbonate, bismuth oxychloride, bismuth trioxide, barium sulfate, tungsten, and mixtures thereof.

In certain embodiments, an implant may also contain pharmacological agents. The pharmacological agent may be, for example, a growth factor to assist in the repair of the endplates and/or the annulus fibrosis. Non-limiting examples of growth factors include a bone morphogenetic protein, transforming growth factor (TGF-β), insulin-like growth factor, platelet-derived growth factor, fibroblast growth factor or other similar growth factor or combinations thereof having the ability to repair the endplates and/or the annulus fibrosis of an intervertebral disc.

In other embodiments of the invention, the pharmacological agent may be one used for treating various spinal conditions, including, for example, degenerative disc disease, spinal arthritis, spinal infection, spinal tumor and osteoporosis. Such agents include, for example, antibiotics, analgesics, anti-inflammatory drugs, including steroids, and combinations thereof.

In a preferred embodiment, the implant is implanted in the spine utilizing a posterolateral, transpedicular approach to the spine, which is lateral to the facet joint and cephalad to the tranverse process of the vertebrae. Such an approach avoids any of the large blood vessels in the body. In certain embodiments, the implant is inserted into a disc space percutaneously.

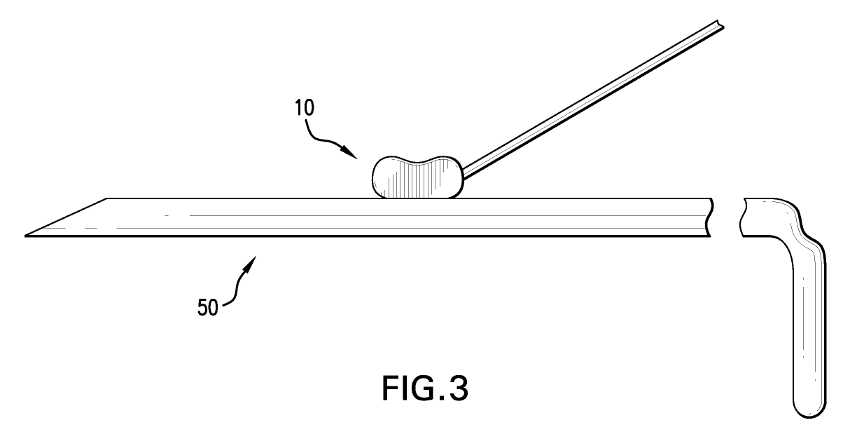

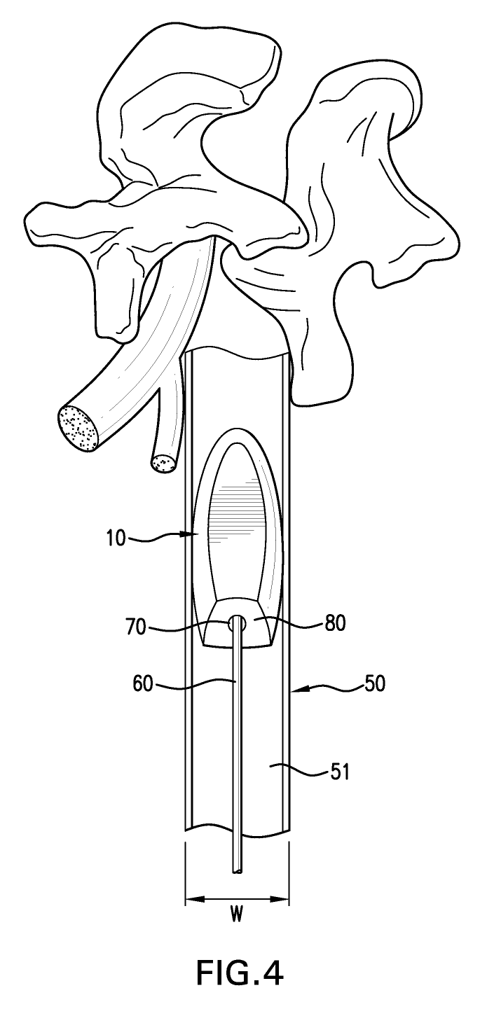

Referring to FIG. 3, in certain embodiments, the present invention provides a kit comprising a nucleus pulposus implant and a trough 50 that can be anchored into a disc space to be repaired and can be used to direct the implant (in this example implant 10) into the intervertebral intra-nuclear location, as seen in FIG. 4. In order to accommodate the implant, trough 50 has a channel 51 with a width W configured to receive the implant. In order to prevent lateral movement of the implant during delivery, preferably channel 51 has a width that closely matches the thickness of the implant. In preferred embodiments, the channel of the trough has a width W of between about 6 mm and 12 mm. In a preferred embodiment, the trough has a width of about 10 mm. In certain embodiments, the width of the channel is between 1 mm and 2 mm greater than the thickness of the implant. Preferably, there is a 1 mm space on each side between the channel and the implant when the implant is placed in the trough.

As seen in FIG. 4, a kit of the present invention can also include a rod 60 to urge implant 10 axially along channel 51 towards the intervertebral intra-nuclear location. In such an embodiment, implant 10 preferably includes a recess or other component 70 integral with a lateral face 80 of implant 10 to mate or otherwise receive the distal end of rod 60 to prevent slippage of the rod from lateral face 80. Lateral face 80 is generally perpendicular or otherwise facing the proximal end of trough 50. Component 70 need not be a recess but can be any other feature that cooperates with the distal end of rod 60 in a male-female relationship to prevent rod 60 from loosing contact with lateral face 80.

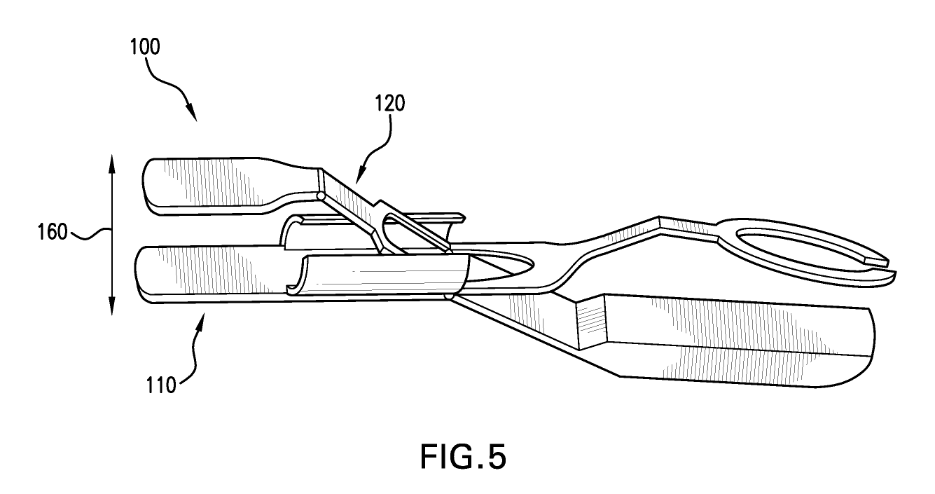

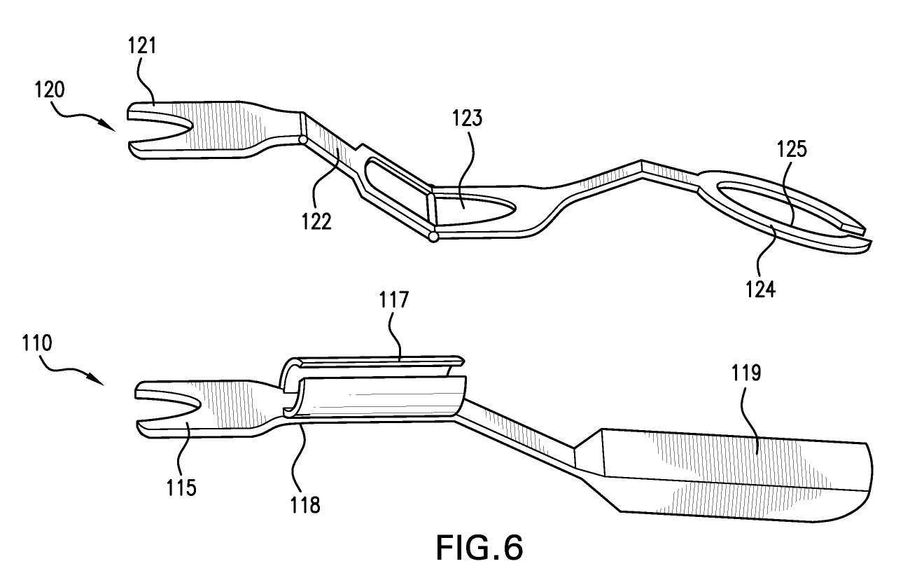

A kit of the present invention can also include an intra-discal distraction device, which distracts the space between the disc to be repaired allowing easier insertion of the implant into the disc space. For example, referring to FIG. 5, an intra-discal distraction device 100 can comprise a first arm 110 pivotably connected to a second arm 120. The distal end of both arms comprises a paddle configured to fit in the intervertebral space and abut against adjacent vertebral endplates. As shown in FIG. 6, where first arm 110 and second arm 120 are separated for the purpose of clarity, first arm 110 has a distal end comprising a paddle 115; an intermediate portion comprising a trough 117, which can optionally be connected (either releasably or irreleasably) to a base member 118; and a proximal portion comprising a handle 119. Second arm 120 has a distal end comprising a paddle 121; an intermediate portion comprising a base member 122 defining an opening 123; and a proximal portion comprising a handle 124 defining a longitudinally extending slot 125.

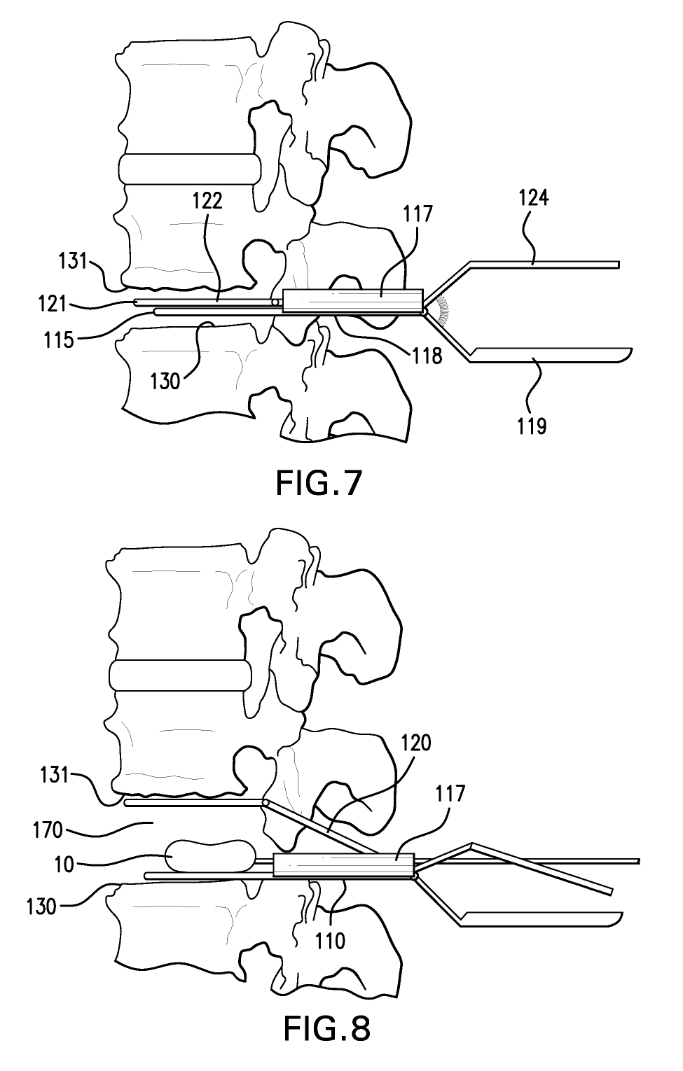

Referring to FIGS. 7 and 8, in use, the intradiscal distraction device 100 is initially in a closed position where base member 122 of the second (or top arm) 120 is disposed in the trough 117 of the first (or bottom arm) 110. Both the first and second paddles 115 and 121 are within the intervertebral space respectively adjacent to opposing first and second vertebral endplates 130 and 131. Pressure is applied to the handles 119 and 124 of the first and second arms to actuate first and second paddles 115 and 121 and to move the paddles substantially along distraction axis 160 (shown in FIG. 6) to an open position corresponding to the desired distracted position of the endplates 130 and 131. The paddles may be moved to a substantially parallel or lordotic position to separate adjacent endplates 130 and 131. Referring to FIG. 8, once the intradiscal distraction device 100 is in an open position, implant 10 can be placed in trough 117 and urged along the longitudinal axis of trough 117 by rod 60 into the intervertebral intra-nuclear location 170. Slot 125 of handle 124 and opening 123 of base member 122 are configured to accommodate the passage of rod 60 therethrough. Distraction device 100 can then be removed. Of course, other variations of the above-described distraction device which incorporates a trough are within the scope of the present invention and the distraction device 100 described above is exemplary

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

I claim:

1. A nucleus pulposus implant comprising:
a sterile kidney-shaped body defining at least one opening on a lateral face of the body, the opening in fluid communication with an internal cavity, the lateral face generally parallel to the horizontal plane of the human body when the implant is in an inserted position in the intervertebral intra-nuclear space.

2. The implant of claim 1, wherein the body of the implant is fabricated from a polymeric material.

3. The implant of claim 2, wherein the polymeric material is high molecular weight polyethylene, polyether ketone, polycarbonate urethane, or combinations thereof.

4. The implant of claim 3, wherein the polymeric material is an elastomeric material.

5. The implant of claim 1, wherein the body is fabricated from a metallic material.

6. The implant of claim 4, wherein the metallic material is cobalt chrome.

7. The implant of claim 1, wherein the implant has the dimensions of a nucleus pulposus.

8. The implant of claim 1, wherein the implant has a height of between about 13 and 18 millimeters.

9. The implant of claim 8, wherein the implant has a thickness of between about 13 and 18 millimeters.

10. The implant of claim 8, wherein the implant has a length between about 22 and 27 millimeters.

11. The implant of claim 1, wherein the opening has a diameter of between about 8 mm and 10 mm.

12. The implant of claim 1, wherein the body of the implant comprises a pharmaceutical agent.

13. The implant of claim 12, wherein the pharmaceutical agent is bone morphogenetic protein.

14. A kit comprising the nucleus pulposus implant of claim 1 and further comprising:
a trough defining a channel extending along a longitudinal axis thereof, the channel having a width configured to receive the implant of claim 1.

15. The kit of claim 14, wherein the channel has a width of between about 6 mm and 12 mm.

16. A kit comprising the nucleus pulposus implant of claim 1 and further comprising a intra-discal distraction device.

17. The kit of claim 14, further comprising an intra-discal distraction device comprising the trough.

18. A method of placing the nucleus pulposus implant of claim 1 into an intervertebral intra-nuclear location comprising:
providing a trough defining a channel extending along a longitudinal axis thereof;
placing the implant in the channel;
urging the implant axially along the channel towards the intervertebral intra-nuclear location;
positioning the implant in the intervertebral intra-nuclear location.

19. The method of claim 18, wherein the intervertebral intra-nuclear location is accessed percutaneously.

20. A nucleus pulposus implant comprising:
a sterile kidney-shaped body defining at least one opening extending therethrough between opposing lateral faces of the body, the opposing lateral faces generally parallel to the horizontal plane of the human body when the implant is in an inserted position in the intervertebral intra-nuclear space.
